(19) United States
(12) Patent Application Publication
(10) Pub. No.: US 2014/0271370 A1
(43) Pub. Date: Sep. 18, 2014

(54) Sterilizer

(71) Applicants: Justin Sirotin, Providence, RI (US); Jeanette Numbers, Providence, RI (US)

(72) Inventors: Justin Sirotin, Providence, RI (US); Jeanette Numbers, Providence, RI (US)

(21) Appl. No.: 13/961,070

(22) Filed: Aug. 7, 2013

Related U.S. Application Data
(60) Provisional application No. 61/680,362, filed on Aug. 7, 2012.

(51) Int. Cl. A61L 2/07

(52) U.S. Cl. CPC A61L 2/07
USPC 422/105; 422/119

(57) ABSTRACT

A sterilizer system includes a base having a chamber configured to hold water, a lid configured to cover the chamber, a switch coupled to the base and in communication with the lid, and circuitry in communication with the switch. The circuitry is configured to determine whether a sterilization cycle was interrupted. The sterilizer system further includes a status indicator configured to indicate whether the sterilization cycle was interrupted based on input from the circuitry.

### TECHNICAL FIELD

The present invention relates to sterilizers, and more specifically to steam sterilizer systems that are used for infant bottles and accessories.

### BACKGROUND ART

Sterilizers are often used to sterilize infant bottles and accessories. In some kinds of sterilizers, such as sterilizers that use steam or boiling water, the parts to be sterilized need to be maintained at a desired temperature for a sufficient period of time in order for the parts to be sterilized. If the sterilization cycle is interrupted in some way, then the parts may not be sufficiently maintained at the desired temperature and may not be sterilized. The sterilizers, however, usually just let the user know when the designated period of time has expired, regardless of whether the sterilization cycle has been interrupted or not. Therefore, the user may not know when the parts have failed to be sterilized.

### SUMMARY OF EMBODIMENTS

In accordance with one embodiment of the invention, a sterilizer system includes a base having a chamber configured to hold water, a lid configured to cover the chamber, a switch coupled to the base and in communication with the lid, and circuitry in communication with the switch. The circuitry is configured to determine whether a sterilization cycle was interrupted. The sterilizer system further includes a status indicator configured to indicate whether the sterilization cycle was interrupted based on input from the circuitry.

In some embodiments, the status indicator may include a series of lights configured to show a status of the sterilization cycle during operation. The status of the sterilization cycle may include a sterilizing period and a cool down period. The status indicator may be further configured to indicate whether the sterilization cycle was successfully completed based on input from the circuitry. The circuitry may include a timer that indicates a period of time for the sterilization cycle. The base may further include a heating element in its lower portion that is configured to heat the water so that steam is formed within the chamber. The system may further include a thermistor in thermal communication with the heating element, and the circuitry may be configured to shut off the heating element based on input from the thermistor. The system may further include a start switch in communication with the circuitry, and the circuitry may be configured to turn on or off the heating element based on input from the start switch. The lid may be pivotably coupled to the base by a hinge mechanism. The hinge mechanism may include one or more springs that bias the lid to an open position. The switch may be a mechanical contact switch, and the contact switch may include a lever arm. The system may further include one or more holders that hold bottles and/or accessories to be sterilized within the chamber.

### BRIEF DESCRIPTION OF THE DRAWINGS

The foregoing features of the invention will be more readily understood by reference to the following detailed description, taken with reference to the accompanying drawings, in which:

FIG. 1 schematically shows a perspective view of a sterilizer system with the lid in a closed position according to embodiments of the present invention;

FIG. 2 schematically shows a front view of the sterilizer system with the lid in the closed position according to embodiments of the present invention;

FIG. 3 schematically shows a back view of the sterilizer system with the lid in the closed position according to embodiments of the present invention;

FIG. 4 schematically shows a side view of the sterilizer system with the lid in the closed position according to embodiments of the present invention;

FIG. 5 schematically shows a bottom view of the sterilizer system with the lid in the closed position according to embodiments of the present invention;

FIG. 6 schematically shows a perspective view of the sterilizer system with the lid in an open position according to embodiments of the present invention;

FIGS. 7 and 8 schematically show bottle and accessory holders, respectively, for a sterilizer system according to embodiments of the present invention;

FIGS. 9A-E show a process flow diagram of various states of the sterilizer system according to embodiments of the present invention;

FIGS. 10 and 11 schematically show the bottle and accessory holders' placement in the sterilizer system according to embodiments of the present invention;

FIG. 12A schematically shows a perspective view of the sterilizer system at the start of a cycle, and FIGS. 12B and 12C show the status indicator during various stages of the cycle according to embodiments of the present invention;

FIG. 13 schematically shows a cross-sectional, perspective view of the sterilizer system, along lines A-A of FIG. 1, showing some inner components according to embodiments of the present invention;

FIG. 14 shows a close-up view of the latch mechanism and switch for the lid shown in FIG. 13 according to embodiments of the present invention; and

FIGS. 15 and 16 schematically show a cross-sectional view of the latch mechanism and switch for the lid, along lines A-A and B-B of FIG. 1, respectively, according to embodiments of the present invention.

### DETAILED DESCRIPTION OF SPECIFIC EMBODIMENTS

Various embodiments of the present invention provide a sterilizer system that informs the user whether a sterilization cycle has been improperly interrupted or successfully completed. The sterilizer system includes circuitry that controls the operation of the system and determines whether the sterilization cycle was interrupted and a status indicator that lets the user know the status of the sterilization cycle during operation, including when the sterilization cycle was interrupted. Details of illustrative embodiments are discussed below.

FIGS. 1 through 6 schematically show a sterilizer system 10 according to embodiments of the present invention. The sterilizer system 10 includes a base 12 that has a sterilization chamber 14 configured to hold water in its lower portion and to hold bottles and/or other accessories in its middle and/or upper portions. The system 10 also includes a lid 16 that covers the sterilization chamber 14. Preferably, the lid 16 is pivotably coupled to the base 12 by a hinge mechanism 18 which allows any water that has condensed on the underside of the lid 16 to flow back into the chamber 14. The lid 16 may also be placed on or coupled to the base 12 in other ways as known to those skilled in the art. The lid 16 may be secured to the base 12 in a closed position, such as shown in FIGS. 1 through 5, with a latch mechanism 20. When the latch mechanism 20 is actuated, the lid 16 moves from the closed position to an open position, such as shown in FIG. 6. The hinge mechanism 18 may be configured such that the lid 16 is biased to the open position. For example, the hinge mechanism 18 may include one or more springs so that the lid 16 automatically moves from the closed to the open position when the latch mechanism 20 releases the lid 16.

The sterilizer system 10 further includes a switch 22 (as shown and described in more detail below with respect to FIGS. 13-16) coupled to the base 12 and in communication with the lid 16. The switch 22 detects whether the lid 16 is in the open or closed position. The system 10 also includes circuitry (not shown), in communication with the switch 22, that controls the operation of the system and determines whether a sterilization cycle was interrupted or successfully completed. In addition, the system 10 includes a status indicator 26 that shows the user a status of the sterilization cycle, e.g., the progress of the sterilization cycle, the successful completion of the cycle, or the interruption of the cycle.

The base 12 may further include a heating element 24 positioned in its lower portion that heats the water once it is added to the chamber 14. The heating element 24 may be any device that is able to heat the water to a sufficient temperature so that steam is formed within the chamber 14, such as an electrical resistance heating element. The system 10 also may include one or more holders 28, such as a bottle rack as shown in FIG. 7 or an accessory basket as shown in FIG. 8, that hold the bottles and/or accessories to be sterilized within the sterilization chamber 14.

FIGS. 9A-E show a process flow diagram of the various states of the sterilizer system 10 when the system is in operation. The process begins at step 100, in which the sterilizer system 10 enters a standby mode when the system is plugged in. At this point, the system is in a start-up state (step 105) ready to perform a sterilization cycle. In operation, a user adds an amount of water to the chamber 14, places the items to be sterilized onto or into the holders 28 and loads the items into the chamber 14, such as shown in FIGS. 10 and 11. The user closes the lid 16 and starts the sterilization cycle (step 110), e.g., by pressing a start button 30, such as shown in FIG. 12A.

Once the sterilization cycle begins (step 120), the system enters a heating state (step 125), in which the heating element 24 heats the water, turning it into steam, in order to sterilize the parts within the chamber 14. At this time, the status indicator 26 informs the user of the status of the sterilization cycle. For example, the status indicator 26 may include one or more lights (e.g., a series of LEDs) that may either light up or turn off showing the progress of the sterilizing cycle. For instance, a series of lights may initially be all lit (as shown in FIG. 12B) and then some lights 26a may incrementally turn off (as shown in FIG. 12C), showing the progress of the sterilization cycle.

If the sterilization cycle is successfully completed, the system 10 begins a cool down period (step 130) and enters a cooling state (step 135) in which the heating element 24 is turned off and the parts within the chamber 14 begin to cool. In step 140, the status indicator 26 may show the user the progress of the cool down period, e.g., in FIG. 12C, the lights 26b may incrementally turn off. If the cool down period is successfully completed, then the system indicates to the user that the entire cycle was successfully completed (step 150) and the system enters a done state (step 155). For example, in FIG. 12C, a status light 26c may turn green to let the user know that the parts are sterilized and ready to be removed from the system. Once the user opens the lid 16, presses the start button 30, or after a specified waiting period, the system 10 returns to the standby mode (step 160), the status light 26c turns off, and the system returns to the start-up state (step 105) ready to perform another sterilization cycle.

If the user opens the lid 16 or presses the start button 30 after the sterilization cycle is complete, but before the cool down period begins (step 170), then the system 10 returns to the standby mode (step 160) and the status light 26c turns off. Similarly, if the user opens the lid 16 or presses the start button 30 during the cool down period (step 180), then the system 10 returns to the standby mode (step 160) and the status light 26c turns off. When the system is in the standby mode, the system returns to the start-up state (step 105) ready to perform another sterilization cycle.

If the system 10 is interrupted during the sterilization cycle (step 190), then the system enters a fault state (step 195), in which the heating element 24 is turned off and the status indicator 26 lets the user know that the sterilization cycle has been improperly interrupted. For example, the status light 26c may blink red to indicate that the parts may not be sterilized. If the user presses the start button 30 (step 200), then the system 10 returns to the standby mode (step 160) and the status light 26c turns off.

The sterilization cycle may be interrupted in a number of ways. For example, the sterilization cycle may not be successfully completed if the lid 16 is opened during the cycle. The sterilization cycle may also be interrupted if an insufficient amount of water is added to the chamber 14. In this case, the water will boil off sometime during the sterilization cycle, and the parts will not be maintained at the desired temperature for a sufficient period of time in order to sterilize the parts. The sterilization cycle could also be interrupted if the start button 30 is pressed during the sterilization cycle, causing the heating element 24 to be turned off and the parts to be not sterilized.

The sterilizer system 10 is able to determine whether the sterilization cycle was interrupted with the use of circuitry that controls the operation of the system. The status indicator 26 then indicates whether the sterilization cycle was interrupted or successfully completed based on input from the circuitry. The circuitry preferably includes a timer that is meant to run the desired duration of the sterilization cycle, e.g., about 8-16 minutes or more. When the sterilization cycle begins, the circuitry starts the sterilization cycle timer and causes the heating element 24, which is in communication with the circuitry, to turn on. When the timer expires, the circuitry causes the heating element 24 to turn off and informs the status indicator 26 that the sterilization cycle has been successfully completed.

During normal operation when sufficient water is present, the operating temperature of the heating element 24 is around the boiling point of the water (around 212° F.). However, if an insufficient amount of water is present causing the water to boil off during the sterilization cycle, then the temperature of the heating element 24 may rise to above a desired operating temperature. To protect the system from this situation, the system 10 may include a temperature control unit (TCO), such as a thermistor, in thermal communication with the heating element 24, that detects the temperature of the heating element 24. If the TCO detects that the heating element 24 is exceeding a designated temperature (e.g., about 260° F. or greater), then the TCO informs the circuitry, which causes the heating element 24 to turn off based on that input. If the circuitry receives the input from the TCO before the sterilization cycle timer has expired, then the circuitry determines that the sterilization cycle was interrupted before the cycle was successfully completed and the circuitry causes the status indicator 26 to indicate that the cycle was interrupted. Once the heating element 24 temperature has dropped, the TCO resets and is ready for the next sterilization cycle.

In the event that the TCO malfunctions in some way, the system 10 may also include one or more thermal cutout units (TCU), such as thermal fuses, which cause the power to the heating element 24 to be turned off if the heating element 24 exceeds a set temperature (e.g., about 340° F. or greater). The TCU protects the system from a meltdown, but once the TCU is activated, the system will no longer work.

As mentioned above, the sterilization cycle may also be interrupted when the lid 16 is opened during the cycle. In this case, the system 10 detects that the lid 16 is in the open position through the use of a switch 22 in communication with the lid 16. The switch 22 may be any type of switch that is able to detect the position of the lid 16, e.g., a mechanical, optical, or electromechanical switch. For example, as shown in FIGS. 13-16, the switch 22 may be a contact switch that includes a lever arm 32.

When the lid 16 is in the closed position, a bottom portion 16b of the lid 16 may contact a top portion of a pin 34, causing it to be pushed down so that the bottom portion of the pin 34 contacts and actuates the lever arm 32. In FIGS. 13-16, the pin 34 is shown as barely contacting the lever arm 32 (or not touching it at all) even though the lid 16 is closed just for convenience in describing the various components and their functions. The pin 34 may be operably coupled to a spring 36 that biases the pin 34 in an upward position. When the lid 16 is closed and the pin 34 is pressed downward, the spring 36 is compressed.

When the latch mechanism 20 is actuated to open the lid 16 (e.g., the latch mechanism may be pivoted to release a portion of the lid 16), the bottom portion 16b of the lid lifts off of the top portion of the pin 34. This allows the spring 36 to return to its uncompressed state causing the pin 34 to move in an upward direction. As the pin 34 moves upward, the bottom portion of the pin 34 no longer contacts the lever arm 34 of the switch 22.

The switch 22 is in communication with the circuitry and informs the circuitry when the lid 16 is opened or closed. If the circuitry receives input from the switch 22 that the lid 16 has been opened before the sterilization cycle timer has expired, then the circuitry determines that the sterilization cycle was interrupted before the cycle was successfully completed. The circuitry then causes the status indicator 26 to indicate that the cycle was interrupted and also causes the heating element 24 to turn off.

The sterilization cycle may also be interrupted if the start button 30 is pressed again during the sterilization cycle. In this case, the circuitry is informed that the start button 30 is pressed (e.g., the start button may actuate a start switch in communication with the circuitry) and causes the heating element 24 to turn off. If the circuitry receives input from the start switch before the sterilization cycle timer has expired, then the circuitry determines that the sterilization cycle was interrupted before the cycle was successfully completed. The circuitry then causes the status indicator 26 to indicate that the cycle was interrupted.

Although the above discussion discloses various exemplary embodiments of the invention, it should be apparent that those skilled in the art can make various modifications that will achieve some of the advantages of the invention without departing from the true scope of the invention

What is claimed is:

1. A sterilizer system comprising:
a base having a chamber configured to hold water;
a lid configured to cover the chamber;
a switch coupled to the base and in communication with the lid;
circuitry, in communication with the switch, configured to determine whether a sterilization cycle was interrupted; and
a status indicator configured to indicate whether the sterilization cycle was interrupted based on input from the circuitry.

2. The sterilizer system of claim 1, wherein the status indicator includes a series of lights configured to show a status of the sterilization cycle during operation.

3. The sterilizer system of claim 2, wherein the status of the sterilization cycle includes a sterilizing period and a cool down period.

4. The sterilizer system of claim 1, wherein the status indicator is further configured to indicate whether the sterilization cycle was successfully completed based on input from the circuitry.

5. The sterilizer system of claim 1, wherein the circuitry includes a timer that indicates a period of time for the sterilization cycle.

6. The sterilizer system of claim 1, wherein the base further includes a heating element in its lower portion, the heating element configured to heat the water so that steam is formed within the chamber.

7. The sterilizer system of claim 6, further comprising a thermistor in thermal communication with the heating element, the circuitry configured to shut off the heating element based on input from the thermistor.

8. The sterilizer system of claim 6, further comprising a start switch in communication with the circuitry, the circuitry configured to turn on or off the heating element based on input from the start switch.

9. The sterilizer system of claim 1, wherein the lid is pivotably coupled to the base by a hinge mechanism.

10. The sterilizer system of claim 9, wherein the hinge mechanism includes one or more springs that bias the lid to an open position.

11. The sterilizer system of claim 1, wherein the switch is a contact switch.

12. The sterilizer system of claim 11, wherein the contact switch has a lever arm.

13. The sterilizer system of claim 1, further comprising one or more holders that hold bottles or accessories to be sterilized within the chamber.
